(19) United States
(12) Patent Application Publication
(10) Pub. No.: US 2005/0049184 A1
(43) Pub. Date: Mar. 3, 2005

(54) Treatments which elevate functional glycosylated leptin trasnport factor, for controlling weight and obesity

(75) Inventors: Hao Qian, St. Charles, MO (US); Ronald Gingerich, St. Charles, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Ronald Gingerich

(21) Appl. No.: 10/938,049

(22) Filed: Sep. 10, 2004

Related U.S. Application Data
(63) Continuation of application No. 09/922,450, filed on Aug. 4, 2001, now abandoned.
(60) Provisional application No. 60/222,813, filed on Aug. 4, 2000.

(51) Int. Cl. A61K 38/17

(52) U.S. Cl. 514/8

(57) ABSTRACT

Methods and compounds for treating obesity and inducing weight loss use a functional, glycosylated leptin transport factor (LTF) polypeptide, referred to as fn/glyLTF. An unstable defective version of the LTF protein, referred to herein as def/LTF, is present in freshly-drawn blood from obese animals or people; it is degraded rapidly in circulating blood. In people with normal body weight, fn/glyLTF stabilizes and protects leptin, a hormone with powerful effects on fat metabolism and body mass. LTF apparently is the same protein previously recognized as a soluble truncated fragment of the obesity receptor (Ob-R) protein, referred to in the prior art as Ob-Re, or sOb-R. In humans with normal body weight, fn/glyLYF has a weight of about 145 kD, compared to a polypeptide-only weight of about 93 kD. defLTF has a substantially lower molecular weight, and tests using deglycosylating enzymes indicate that it is not glycosylated to the same level as fn/glyLTF. Treatment methods include: (1) elevating concentrations of fn/glyLTF in circulating blood, by means such as intravenous injection or sustained-release implants, or by gene therapy; (2) suppressing enzymatic deglycosylation in circulating blood, such as by extracorporeal removal of deglycosylating enzymes; and, (3) providing “surrogate” forms of fn/glyLTF. Diagnostic kits are also disclosed, for measuring both fn/glyLTF and def/LTF in animals and people suffering from obesity.

### RELATED APPLICATION

This application claims the benefit under 35 USC 119(e) of provisional application 60/222,813, filed Aug. 4, 2000.

### BACKGROUND OF THE INVENTION

This invention is in the fields of medicine and pharmacology, and relates to a natural hormone called leptin, which affects body weight and fat metabolism.

The physiological roles of leptin and leptin receptors are discussed in review articles such as Spiegelman et al 1996, Considine et al 1996, and Friedman et al 1998 (full citations are provided below), and in numerous articles cited therein. Very briefly, leptin is a protein that is encoded by a gene called “ob” (short for “obese”). It was first isolated and identified in 1994, based on genetic analysis of “ob/ob” mice that were grossly overweight due to a mutant ob gene (Zhang et al 1994; the double “ob/ob” designation indicates that both chromosomal copies of the ob gene, in the somatic cells of these mice, were mutant forms).

DNA sequence data for the leptin gene, and amino acid sequence data for the leptin protein, have been published for both the mouse version and the human version, in articles and patents such as Zhang et al 1994 and Tartaglia et al 1995. The human protein reportedly has 84% homology with the mouse protein.

Leptin is a hormone. With the help of certain other molecules discussed in more detail below, it is transported across the blood-brain barrier, into the brain. After it enters the brain, it plays a crucial role in a complex multi-part feedback system that helps balance two fundamental goals. First, this system allows and helps animals to accumulate surplus energy stores, in the form of fat, when food is abundant. And second, when it functions properly, this system helps animals control their weight and burn off excess fat, so that they will not become obese even if they eat too much for prolonged periods of food surplus.

To a large extent, the feedback and control system depends on a crucial mechanism: leptin is generated by “adipose” (fatty) cells and tissue. Therefore, as an animal accumulates more fat, the adipose cells within that animal's fatty storage tissue will generate larger quantities of leptin, which will enter circulating blood. When the system works properly, higher concentrations of leptin in circulating blood will cause greater quantities of leptin to enter the brain.

After leptin enters the brain, it exerts several powerful effects. It apparently suppresses appetite, leading to a reduction of additional food intake. The exact mechanism(s) are not fully understood, but may involve inhibiting the expression, activity, or other traits of hormones or neurotransmitters which have “orexigenic” (appetite stimulating) effects and which are present in high quantities in obese animals; orexigenic compounds include neuropeptide Y and possibly various other compounds, such as melanin concentrating hormone, galanin, orexin, and Peptide YY, as reviewed in Spiegelman et al 1996 and Friedman et al 1998.

Various reports also suggest that leptin stimulates energy expenditure. In ob/ob mice, administration of exogenous leptin reportedly led to increases in body temperature, oxygen consumption, and locomotor activity (Pelleymounter et al 1995; Halaas et al 1995; Schwartz et al 1996). Increases in body temperature following injection of leptin were also reported to be independent of levels of physical activity (Collins et al 1996).

Regardless of the specific molecular or cellular mechanisms involved, it is clear that the effects of elevated concentrations of leptin inside the brain (including reduced appetite and food intake, and possibly increased energy expenditure) can contribute to processes which lead to burning off some of the accumulated fat in an animal's body, and a reduction of the weight of the animal. Accordingly, when the leptin system functions properly, it helps an animal stay healthy and vigorous, with a reasonably stable and constant weight, despite large fluctuations in its food supply.

However, if the leptin system fails to work properly, it leads to unwanted weight gain, and eventually to obesity. An animal model of a defective leptin system is provided by “ob/ob” mice, which have two copies of a defective, nonfunctional “ob” gene, resulting in a dysfunctional leptin hormone. When fed the same diet as normal mice, they accumulate 5 times as much fat, and their total body weight bulks up to 3 times the total body weight of healthy mice (Friedman et al 1998; Coleman 1978).

The ability of leptin to cross the so-called “blood-brain barrier” (BBB) and enter brain tissue deserves further attention. The BBB is not a single membrane; instead, it arises from the fact that, inside the central nervous system (CNS, which includes the brain and spinal cord, and a few other types of tissue which are not relevant herein, such as retinas), the walls of capillaries have a “tighter” structure than the walls of capillaries in other types of tissue outside the CNS. The BBB prevents a wide range of molecules (including most proteins and amino acids) from entering brain tissue, unless they are carried across the BBB by some form of active transport mechanism.

Since leptin is a protein, it is strongly presumed and inferred that some type of active transport system causes leptin to be transported across the BBB. However, under the prior art, very little is known about the transport system involved in leptin transport.

Proper functioning of the leptin hormone depends on a set of proteins that are usually called “leptin receptor” proteins. In both mice and humans, there are five known types of leptin receptor proteins, having different lengths. These proteins have been given the names Ob-Ra, Ob-Rb, Ob-Rc, Ob-Rd, and ob-Re, where “Ob-R” stands for “obesity receptor”, and the “a” through “e” designations were assigned arbitrarily as each new variant was isolated and identified.

All five of these variant forms are believed to be encoded by a single gene, which is designated as the “db” gene. The “db” designation is short for “diabetes”. This gene was initially isolated and identified from overweight mutant “db/db” mice which exhibited traits that are similar to diabetes in humans. Scientists had not realized, during that early stage of research, that the protein generated by the “db” gene has nothing to do with insulin, and instead relates to leptin receptor proteins.

As mentioned above, all five different forms of the leptin receptor protein are believed to be derived from a single gene. The reasons for the variations in these proteins are not fully understood, but are presumably due to factors such as (i) differential splicing mechanisms of the messenger RNA (several distinct cDNA's have been identified, as reviewed in Considine et al 1996); and/or, (ii) differences in post-translational processing, such as enzymatic cleavage of a long initial polypeptide to generate shorter polypeptides.

The longest form of the leptin receptor protein is designated as Ob-Rb. The mouse version of this protein has 1162 amino acids. Like nearly all receptor proteins, it straddles a cell membrane. Part of the protein, a strand having about 800 amino acids (including the amine terminus) is positioned outside the cell. This extracellular portion is exposed to leptin that circulates in extracellular fluid. A short segment rests inside the cell membrane, effectively anchoring the protein to the membrane. The remainder of the protein strand (the intracellular portion, with about 350 amino acids, including the carboxy terminus) remains inside the cell.

The Ob-Ra, Ob-Rc, and Ob-Rd variants have shorter lengths, ranging from 892 to 900 amino acids (in mice). All of these versions are believed to remain anchored to the cell membrane as well. They have fully intact extracellular domains, and the portion that has been truncated is the intracellular segment.

The protein that has been designated as Ob-Re is the shortest known Ob-R variant, with 805 amino acids in a mice version that was analyzed (Friedman et al 1998), and 818 amino acids in a human version that was analyzed (Haniu et al 1998). Importantly, the “e” form of the Ob-R protein is not anchored to a cellular membrane at all; instead, it is secreted in soluble form, and it circulates freely in blood; for that reason, it is referred to in some articles as “sOb-R”, where the “s” prefix refers to “soluble”.

The soluble “e” form of the Ob-R receptor protein is known to be glycosylated. In layman's terms, a “glycosylated” protein has relatively large numbers of sugar molecules (also called saccharide rings, or carbohydrate groups) bonded to the protein. Many types of proteins are glycosylated, and glycosylation is an important and well-known natural process; summaries are contained in nearly any textbook on biochemistry, molecular biology, and medical physiology (e.g., Alberts et al 1994), and in numerous review articles. Based on calculations and on measurements of Ob-Re after it has been treated with deglycosylating enzymes that will cleave off the sugar groups, the polypeptide portion of human Ob-Re has a molecular weight of about 93,000 daltons, while the glycosylated form has a molecular weight of about 145,000 daltons (Hanui et al 1998). Accordingly, the sugar rings make up about 36% of the weight of the glycosylated form of Ob-Re. In mice, the Ob-Re protein is somewhat smaller (reportedly 805 vs. 818 amino acids), and has a molecular weight of about 120,000 daltons, as indicated by migration through gels.

Research has indicated that the human “homologs” of the extensively studied mouse and rat leptin and leptin receptor proteins function in the same or very similar manners. For example, obese humans have abnormally high levels of leptin in circulating blood (Considine et al 1996; Montague et al 1997). In addition, an inherited familial line of human obesity was discovered which appears to be directly attributable to a defective mutant version of the leptin receptor (OB-Rb) gene (Clement et al 1998).

However, obvious defects in either ob or db genes or proteins of obese humans are surprisingly rare, considering how many people suffer from obesity. Most obese people who have been genetically analyzed to date appear to have entirely normal ob and db genes. Therefore, prior to this invention, researchers have not been able to determine certain key components and steps in the highly complex puzzle of the leptin feedback and control system. For example, the review article by Friedman et al, published in late 1998, contains at least a dozen passages which explicitly point out areas of uncertainty, as targets for subsequent research. As examples, Friedman et al 1998 contains the following statements:

“Although the Ob-Ra isoform . . . is expressed in the choroid plexus and many other tissues, its significance is unknown. Ob-Ra can activate gene expression and signal transduction in cultured cells, albeit weakly. It is unknown whether this occurs in vivo. The function of the other forms [Ob-Rc, Ob-Rd, and Ob-Re] is likewise unclear. They may function in the transport of leptin across the blood-brain barrier or form heterodimers with other cell-surface proteins . . . ”

“The attenuation of the leptin response may be explained by the presence of other, undiscovered, signals, perhaps from skeletal muscle. These results also indicate that the effects of recombinant leptin are qualitatively different from those seen after parabiosis in which lean mice receiving db/db (hyperleptinaemic) plasma become anorectic and die of apparent starvation. A factor(s) other than leptin may be required for lethality after parabiosis of wild-type mice to db mice . . . ”

“The mechanisms of leptin transport into the CNS is unknown. As leptin uptake occurs in the capillary endothelium of mouse and human brain, active transport by Ob-Ra or other proteins has been suggested as a possible mechanism . . . ”

Clearly, as described above, researchers have been unable to figure out one or more apparently crucial pieces in the puzzle of the leptin system, which plays a crucial role in weight control.

The Inventors herein have discovered an important part of that puzzle, for human obesity. The data presented below indicate that the so-called “obesity receptor E” protein is not (or is not only) a receptor protein, in the normal sense; alternately or additionally, it functions as a “leptin transport factor” which facilitates the transport of leptin molecules to and/or across the mammalian blood-brain barrier. Accordingly, the soluble form of the so-called “Ob-Re” protein is referred to herein as the “LTF” protein, where “LTF” is an acronym for “leptin transport factor”. Based on the data reported herein, it appears that properly functioning leptin transport factor (LTF) proteins, referred to as the OB-Re or sOb-R receptor protein in published articles, can greatly increase the quantity of leptin molecules which permeate through the BBB and actually enter brain tissue, where they can exert their normal and proper hormonal effects in controlling energy metabolism, fat metabolism, and body weight.

Even more importantly, the Inventors have discovered that, in at least some obese animals and humans, the LTF (or Ob-Re, or sOb-R) protein exists in a very different form than is found in animals or humans having normal body weight. The properly functioning version, which is found in normal quantities in the blood of healthy people with normal body weight, is a larger molecule with a substantially heavier molecular weight. By contrast, the second form is a smaller molecule with a substantially lower molecular weight, and it is relatively unstable; to the extent that it can be detected, it appears mainly in freshly drawn blood from obese people. However, it disappears relatively quickly, in blood which has been stored for a substantial length of time. This indicates that it is relatively unstable and is degraded fairly quickly, presumably by hydrolytic or other enzymes that naturally exist in circulating blood.

Due to their differences in molecular size and weight, these two different version of LTF show up as distinctly different “bands”, when separated on various types of gels that are used to separate proteins. As a shorthand notation, the functional, heavier, stable version of LTF is referred to herein as “fn/glyLTF”. By contrast, the defective, lighter, unstable version is referred to as “def/LTF”.

Although the extent and role of “glycosylation” has not yet been definitively evaluated and proven, the results of certain lab tests performed to date (described below) suggest that glycosylation may play an important and possibly crucial role in the difference between fn/glyLTF, which is found in blood from healthy people with normal body weight, and def/LTF, found in blood from people suffering from obesity.

Because of certain test results described below, the heavier and properly-functioning desirable form of the LTF protein is believed and presumed to be glycosylated to a fairly extensive level. By contrast, the lighter, defective, unstable form of the LTF protein is believed and presumed to have far fewer sugar moieties bonded to the protein.

Accordingly, the defective form is presumed and believed to be a “deglycosylated” protein; this term implies that, in obese animals and humans who suffer from this defect, either or both of two things happened to the defective def/LTF protein.

First, the LTF protein may have never been properly glycosylated, during the process of normal protein formation and glycosylation (both of these processes normally occur inside cells, before a protein is secreted). This type of never-glycosylated protein might be referred to as a “non-glycosylated” protein if desired, since the “de-” prefix often tends to imply that something was initially present but has been removed. However, for convenience, a never-properly-glycosylated protein is referrred to herein as a deglycosylated protein, since the term “deglycosylated” is used more commonly among biochemists than the term “nonglycosylated”. The failure of glycosylation to occur properly inside a cell, before a protein is secreted, can be due to any of several mechanisms. For example, in some patients who suffer from obesity, the glycosylation site itself in a protein might be mutated, in a way that renders it ineffective as a glycosylation site. Haniu et al 1998 reported that two N-glycosylation sites appear to exist in the soluble portion of the “db” gene product; both glycosylation sites contain a “WSXWS” sequence, where W refers to a tryptophan residue, S refers to a serine residue, and X is a variable. According to Haniu et al, these WSXWS glycosylation sites occur at residues 319-323 and at residues 622-626 of the human sOb-R sequence. Accordingly, animals or humans suffering from obesity can be genetically analyzed, to determine whether they have a mutation at or near either or both of those two sites in their db gene sequence.

A second common and likely problem that can lead to deglycosylated LTF molecules is this: after a polypeptide molecule has been fully and properly glycosylated, some or all of the sugar moieties can be stripped away from it, by one or more deglycosylating enzymes that are in an over-abundant or hyper-active state in animals or people suffering from obesity. Such deglycosylating enzymes may exist inside cells, where they may attack and degrade glycosylated LTF before secretion by the cells, or they may exist in circulating blood, where they will attack and degrade glycosylated LTF after secretion by the cells.

Although deglycosylation is a primary candidate which is believed to help explain some or all of the differences between the properly-functioning heavier version of fn/glyLTF and the defective lighter version of def/LTF, the possible role of deglycosylation has not yet been evaluated to a level which (i) establishes a scientific consensus or certainty, and (ii) excludes other possible mechanisms as alternative causative factors or additional aggravating factors. It may be that one or more other mechanisms (such as protein cleavage or other digestion, which might be caused or aggravated by hyperactive hydrolytic or other digestive or degradative enzymes in obese animals and people, or a failure of the various LTF-forming machanisms to create the proper full-length LTF protein in the first place) may also be involved, as causative and/or aggravating factors, in the system defect which prevents or interferes with the creation, secretion, or stability of properly-functioning LTF molecules in at least some people who suffer from obesity due to defects in their LTF system. For example, as one scenario that can and should be evaluated, if a substantial portion of either end (including the amino terminus, or including the carboxy terminus) of the LTF polypeptide sequence is missing, in a way which causes the truncated and lighter protein, even when properly glycosylated, to migrate on gels in a manner comparable to deglycosylated LTF, then the missing end might provoke accelerated rates of proteolytic degradation, due to (for example) the exposure of a cleavage sequence (which otherwise would remain protected within the interior of the protein) to any of various proteolytic enzymes that circulate in blood.

It also should be recognized that different types of defects may be present in different patients. In some obese patients, deglycosylation of def/LTF may pose a primary and crucially important defect, analogous to a broken link in a chain that can no longer function properly. In other obese patients, deglycosylation may play a less important role which merely aggravates another primary problem; or, deglycosylation may not even be involved at all, in some patients who suffer from some other type of defect in their LTF formation and secretion system.

Nevertheless, this discovery and disclosure by the Inventors focuses a powerful spotlight on what appears to be a crucially important factor in a major type of defect in the leptin system, in at least some obese people and animals. The tests and data disclosed herein identify and highlight a particular problem that has been discovered and shown to exist in the leptin transport system of obese animals and people. In addition, this discovery clearly suggests and points to several potential therapeutic interventions, for obese patients who suffer from this particular type of impairment in their leptin regulatory control system.

Accordingly, one object of this invention is to disclose that the leptin system requires and depends upon a stable and properly-functioning form of a leptin transport factor (LTF) protein, which has a substantially higher molecular weight than a defective, lighter, unstable form of that same LTF protein (which apparently is identical to the protein referred to in the prior art as an obesity receptor protein (Ob-Re)).

Another object of this invention is to disclose certain test data which suggest that the defective, lighter, unstable form of LTF that is found in at least some obese people and animals may be caused by either or both of the following: (i) deglycosylation of properly-glycosylated fn/glyLTF molecules, after they have been secreted by cells; and/or (ii) a failure of the LTF-glycosylation mechanism, which prevents LTF polypeptides from being properly synthesized, glycosylated, and secreted by cells.

Another object of this invention is to disclose therapeutic interventions for obese patients whose blood contains the defective, lighter, unstable form of LTF. Such interventions include: (i) administration of properly glycosylated fn/glyLTF molecules, using methods such as intravenous or intramuscular injection; (ii) prolonged-release administration of fn/glyLTF molecules, using implantable devices such as minipumps, osmotic diffusion devices, or resorbable matrices; (iii) “direct” genetic engineering, using vectors that are introduced directly into a patient's body; (iv) implantation of cells that have been taken from a patient and subjected to genetic engineering, to establish or increase their expression of enzymes which increase the expression or stability of fn/glyLTF polypeptides; (v) implantation of “exogenous” cells (i.e., cells derived from any source other than a patient's own body) which synthesize and secrete fn/glyLTF, and which can immunosequestered if desired to protect them from a patient's immune system; and, (vi) administration of chemical compounds which can act as regulators to increase the production of fn/glyLTF, or to suppress the degradation of fn/glyLTF.

These and other forms of treatment will become more apparent from the following summary and description of the preferred embodiments.

### SUMMARY OF THE INVENTION

This invention discloses a method for treating obesity and providing improved pharmaceutical control over body weight. This invention is based on the discovery that, while a “leptin transport factor” (LTF) protein exists in a relatively stable glycosylated form referred to herein as fn/glyLTF, in animals and people with normal body weight, a substantially smaller and unstable version of the LTF protein, referred to herein as def/LTF, exists in the blood of obese animals and people. The smaller and unstable def/LTF is quickly degraded once it enters the circulating blood of obese animals and people. The LTF protein plays a major role in stabilizing and protecting leptin (a hormone that exerts powerful effects on fat metabolism and body mass) in the circulating blood. The LTF protein helps blood-borne leptin reach the brain, pass through the blood-brain barrier, and exert its hormonal effects inside CNS tissue.

The protein referred to herein as LTF apparently is the same protein that was previously recognized as a soluble truncated version of the obesity receptor (Ob-R) protein, which normally is embedded in the membranes of cells. In the prior art, the truncated part of the membrane receptor protein which is found in soluble form in circulating blood is usually designated as Ob-Re, or as sOb-R. It has slightly over 800 amino acid residues in both the mouse and human forms, and is believed to be encoded by the same “db” gene that encodes the complete membrane-embedded Ob-Rb receptor protein.

The stable and functional version of fn/glyLTF which is found in animals and people having normal body weight is glycosylated, to a level which converts its polypeptide-only molecular weight of about 93,000 daltons (human form) to a total of about 145,000 daltons. By contrast, the defective and unstable version of LTF that can be found in freshly-drawn blood from obese animals and people has a substantially lower molecular weight. Various tests (including tests using deglycosylating enzymes) indicate that this unstable LTF protein, called def/LTF, is not glycosylated at the same level which occurs in stable and functional LTF, as found in people with normal body weight. This apparent absence of normal glycosylation in def/LTF is presumed to be due to either: (i) a defect in the glycosylation mechanism which occurs inside cells that normally should synthesize and secrete properly-functioning glycosylated LTF into circulating blood; and/or, (ii) excessively high activity by one or more deglycosylating enzymes, which strip away the glycosyl moieties from the LTF polypeptide after the LTF polypeptide enters circulating blood.

The discovery that unstable def/LTF (as found in freshly drawn blood from obese animals and humans) is substantially different from fn/glyLTF, which is much more stable and long-lasting in the blood of healthy animals and humans, indicates that any of several therapeutic interventions can be developed and used for treating obese patients who suffer from a defect in their LTF system. Such interventions can generally be grouped into three major categories: (1) methods and compounds for directly elevating concentrations of fn/glyLTF in circulating blood; (2) methods and compounds for suppressing enzymes that deglycosylate fn/glyLTF or otherwise hasten the degradation of fn/glyLTF in circulating blood; and, (3) methods and compounds which can function as “surrogate” forms of fn/glyLTF.

This invention further discloses methods of using fn/glyLTF in circulating blood as an indicator compound, for use in analyzing and diagnosing factors which contribute to impairments in fat metabolism and weight control, in obese patients. In particular, this invention includes the development of immunoassays (including radioimmunoassays, ELISA assays, etc.) and analytical techniques (including Western blotting or other electrophoretic, chromatographic, or microarray techniques) for analyzing fn/glyLTF and def/LTF concentrations in blood, cerebrospinal fluid, or other body fluids or tissues.

This invention also discloses the development of monoclonal or polyclonal antibody lines which can distinguish between fn/glyLTF and def/LTF. For example, such antibody reagents might include two distinct antibody types, one of which binds selectively to fn/glyLTF but not to def/LTF, while the other type binds selectively to def/LTF but not to fn/glyLTF. Alternately, such antibody reagents can include two distinct antibody types, one of which binds nonselectively to both fn/glyLTF and def/LTF, while the other binds selectively to either fn/glyLTF or def/LTF (but not both).

Finally, this invention also discloses preparations, reagents, testing kits, and methods which can specifically distinguish between fn/glyLTF and def/LTF, for use in testing “fa/fa” rats or other animals, or for testing blood or tissue from obese humans. Such kits and reagents offer highly useful tools and reagents for medical analysis that focuses specifically on the roles of fn/glyLTF and def/LTF in fat metabolism, energy metabolism, and weight control.

### BRIEF DESCRIPTION OF THE DRAWINGS

FIG. 1 shows bands of glycosylated LTF (at about 120 kilodaltons) and non-glycosylated LTF (at about 70 kilodaltons) from blood serum taken from lean rats (lanes 1-4) and obese “fa/fa” rats (lanes 5-8), processed using denaturing gel electrophoresis, followed by Western blotting using an antibody preparation that binds to both fn/glyLTF and def/LTF. These bands show that lean rats have substantially higher levels of fn/glyLTF in their blood.

FIG. 2 compares bands of LTF (from lean and obese rats) that have been treated by a deglycosylating enzyme called PNGase. Using blood serum from lean rats, deglycosylating treatment (lanes 1-2) caused a major shift downward in the LTF bands, compared to untreated controls (lanes 3-4). By contrast, in blood serum from obese rats, the deglycosylating enzyme (lanes 5-6) caused only a minor shift downward in the LTF bands, compared to untreated controls (lanes 7-8). These results support the assertion that in obese animals, LTF is not glycosylated, and is not affected by deglycosylation, while in lean animals, LTF is glycosylated and is sharply affected by deglycosylation.

FIG. 3 shows bands resulting from blotting using both anti-leptin antibodies (bands 1-2 for obese rats, bands 3-4 for lean rats), and anti-LTF antibodies (bands 5-6 for obese rats, bands 7-8 for lean rats). Lanes 1 and 2 show large quantities of free unbound leptin (about 16 kilodaltons), in blood from obese rats, and no detectable levels of unbound leptin in blood from lean rats. Lanes 5 and 6 show no detectable bands of leptin-LTF bound complex, compared to heavy bands in lanes 7 and 8 from lean rats.

FIG. 4 shows the results of time-dependent digestion of fn/glyLTF using the PNGase deglycosylation enzyme for 9 hours (lanes 1-2), 6 hours (lanes 3-4) or 3 hours (lanes 5-6), compared to untreated controls (lanes 7-8). All of these tests used blood from lean rats. The faintness of the deglycosylated LTF bands in lanes 1-2 (9 hour digestion) compared to the heavier bands in lanes 3-4 (6 hour digestion) indicates that LTF is relatively unstable, if it is not protected by the glycosylation moieties.

FIG. 5 shows the results of binding of radiolabelled leptin to LTF proteins from obese rats (lanes 4-7) and lean rats (lanes 8-11). Comparison of these bands shows that the predominant form of LTF in obese rats is the non-glycosylated form, while the predominant form in lean rats is the glycosylated form.

FIG. 6 shows LTF bands from humans, divided into obese patients (lanes 1-2) and lean volunteers (lanes 3-4). These bands show that lean humans have substantially greater levels of fn/glyLTF than obese patients.

FIG. 7 shows LTF bands from human blood serum samples that were kept refrigerated for a month (lanes 1-2), compared to blood serum samples that were kept frozen at −20° C. until shortly before processing (lanes 3-4). The absence of any lower bands in lanes 1-2, as seen in lanes 3-4, provides further evidence that non-glycosylated LTF is relatively unstable in human blood, and is degraded or digested into metabolic waste.

FIG. 8 shows bands from serum taken from lean humans (lanes 3-6) and obese humans (lanes 7-10). These bands were generated by binding of radiolabelled leptin to LTF molecules in the serum from the patients. Comparison of these bands shows that non-glycosylated LTF predominates in obese humans, while glycosylated LTF predominates in lean humans.

### DESCRIPTION OF THE PREFERRED EMBODIMENTS

This invention discloses methods and compounds for treating obesity, and for providing improved pharmaceutical control over body weight. This method can be used medically, for treating human patients; it can also be used by veterinarians, for treating cats, dogs, horses, or other animals that are badly overweight.

This invention is based on the experimental data which indicate that glycosylation plays a highly important role in creating stable and properly-functioning forms of a leptin transport factor (LTF). Without glycosylation, the LTF polypeptide appears to be rapidly degraded (apparently by one or more enzymes in circulating blood) and disappears. If functional glycosylated copies of the LTF protein are not present in circulating blood in adequate numbers, leptin (a powerful hormone) is unable to enter the brain in sufficient quantity, and cannot perform its normal and healthy roles in weight control and energy metabolism.

The LTF transport factor apparently is identical to a polypeptide known in the prior art as OB-Re (an acronym for obesity receptor, “e” form). This “e” form of the obesity receptor polypeptide is believed to be a soluble truncated form of the much longer “b” (OB-Rb) polypeptide, which normally straddles cell membranes and does not circulate in blood. Five different versions of the obesity receptor polypeptide (known as OB-Ra though OB-Re), all having different lengths, are believed to be encoded by a single gene known as the “db” receptor gene (which was named incorrectly, based on an early belief that it was involved in diabetes).

The short form of that protein, known in the prior art as OB-Re but referred to herein as LTF since it has been discovered to be a leptin transport factor, is believed to be formed by post-translational processing (including cleaving and glycosylation) of the substantially longer OB-Rb polypeptide.

As noted above, this invention focuses on the discovery that-glycosylation appears to plays a crucial and essential role in creating and protecting stable properly-functioning copies of the LTF protein. Accordingly, a glycosylated copy of the LTF protein is referred to herein as fn/glyLTF. By contrast, a copy that does not have adequate glycosylation to render it stable and functional is referred to as def/LTF.

Based on that discovery, therapeutic interventions are disclosed for obese patients and other patients who suffer from impaired control over their body weight, and especially for patients who have been diagnosed by blood tests and found to have inadequate levels of fn/glyLTF in their blood.

Such interventions and treatments can be grouped into three major categories, which are listed in the Summary, above, and which are divided into separate subheadings below.

Methods and Compounds for Directly Elevating fn/glyLTF

Several approaches offer highly promising candidate methods for directly increasing concentrations of fn/glyLTF in circulating blood. These approaches include the following:

1. “Short-term” administration of stable and functional fn/glyLTF molecules into circulating blood, using methods such as intravenous injection or infusion (an injection or infusion regimen that lasts a week or less is referred to herein as “short-term”). Other forms of short-term injection, such as intramuscular or subcutaneous injection, can also be evaluated if desired.

As noted above, obese people tend to have extremely high levels of the leptin protein (generated by adipose tissue, which is present in very high quantities in obese people) circulating in their blood. Accordingly, direct intravenous injection or infusion of fn/glyLTF is likely to have profound short-term effects on various metabolic activities and levels, and it should be done only in a hospital or clinical setting, under the supervision of a qualified physician.

2. Sustained-release administration of fn/glyLTF molecules, using implantable devices such as minipumps, osmotic diffusion devices, or resorbable matrices. Depending on the status and needs of a patient, release of the fn/glyLTF drug by an implanted device might last for an “intermediate term” (such as about a week, up to about a month), or a “long term” (with more than a month of continuous fn/glyLTF release). The rate(s) and total quantity released will be controlled accordingly, also based on the status and needs of a specific patient.

If desired, an implanted sustained-release device which holds fn/glyLTF can be provided with replenishment capability. For example, a semi-enclosed reservoir can be provided with a rubber or flexible polymer membrane on one side of the device, and a hypodermic needle and syringe can be used to inject an additional quantity of fn/glyLTF into the reservoir on an as-needed basis (such as each time a patient's weight rises to a level which is higher than a pre-determined unhealthy level). Alternately or additionally, an implanted sustained-release device which holds fn/glyLTF can be provided with a mechanism to stimulate higher short-term rates of drug release, such as by means of magnetic objects or particles that will oscillate, rotate, or otherwise move in response to a magnetic field that can be applied using an external controller.

Resorbable matrices (also called digestible or degradable matrices) can be made of natural fibers (such as collagen, which is gradually digested by collagenase enzymes) and/or various known types of synthetic fibers. These can be formed into three-dimensional porous shapes by means such as freezing a thick slurry in water or other solvent, followed by lyophilizing (“freeze-drying”) under vacuum to remove the solvent while preserving the shape of the article.

3. It should be recognized that intravenous injection or infusion of fn/glyLTF are not the only methods of introducing fn/glyLTF into circulating blood. Various other modes of administration which are well-known to those skilled in the art may also be suitable, as can be evaluated by routine tests. As example, capsules which contain fn/glyLTF inside a coating of keratin or other material which will not be dissolved by stomach acid, but which will be digested once inside the intestines, can be evaluated for use as described herein. Alternately or additionally, trans-membrane routes (such as nasal sprays, skin patches, troches, etc.), rectal suppositories, or any other known mode of administration which can be used to successfully introduce a protein into circulating blood can be evaluated, using no more than routine experimentation. Nevertheless, direct intravenous administration is the most efficacious route, and generally should be used at least during early-stage testing of this treatment method.

4. Gene therapy using autologous cells (i.e., cells which are removed from a patient, manipulated outside the body, and implanted or injected back into the body). Such manipulation typically involves controlled genetic engineering, in which one or more specific known genes (possibly including one or more copies of the gene which encodes the LTF polypeptide sequence, and/or genes which may encode glycosylating enzymes, such as N-glycosidase F) are inserted into a selected cell type. “Blast” or “stem” cells are most commonly used, since they are non-cancerous cells which are capable of reproducing more cells indefinitely; however, “transformed” cells are also used, and in some situations can be subjected to various types of drug treatment to activate or suppress the inserted genes. Descendant cells are subsequently analyzed to identify and isolate cells in which the desired gene(s) have/has been integrated into the cell genome in a stable and functional manner.

5. Gene therapy using heterologous cells (i.e., cells which were originally obtained from a source other than the patient). When such cells are used, they often must be encapsulated within permeable devices or gels which function as “immuno-sequestering” enclosures, such as described in U.S. Pat. Nos. 6,054,142 and 6,231,879 (Li et al, 2000 and 2001) and 5,773,286 (Dionne et al, 1998), to prevent the patient's immune system from attacking the implanted cells. However, various other techniques have been developed, including: (i) using cells that have been genetically engineered to reduce the numbers and types of potentially antigenic proteins on their surfaces; (ii) drug treatments to achieve partial immunosuppression; (iii) implanting cells into certain portions of the body which are effectively immuno-sequestered; and (iv) chemical or electrical methods of fusing two different types of cells together, as used to create “hybridoma” cells, which generate monoclonal antibodies. When cell fusion methods are used, the progeny cells are screened in an effort to identify one or more cell lines which have almost all of their chromosomes from the patient's cells, and have only one or a few chromosomes (including the desired passenger gene) from the foreign cell line.

6. “Direct” genetic therapy, using a vector such as a disarmed (non-pathogenic) virus to insert a “passenger” gene directly into one or more types of target cells inside a patient's body. Disarmed adenoviruses have been widely tested as vectors in this type of direct genetic therapy.

When gene therapy is used, regardless of what mode of introduction is used, the newly-added (exogenous) gene should be selected so that it will have the best chance of overcoming the specific problem that has arisen in a particular patient. When gene therapy is used to treat a patient suffering from a defect in one or more glycosylating mechanisms, the therapy typically will involve introducing one or more exogenous genes which can “fill a gap” that was left vacant when a defective or non-present enzyme was unable to carry out a necessary step in the glycosylation, cytoplasmic transport, and secretion process.

7. Identification and administration of drugs or other compounds that can stimulate the expression of genes which are involved in creating higher quantities of fn/glyLTF. Such drugs might include compounds that have direct “gene activating” effects, and/or drugs that may be involved in one or more feedback circuits which instruct cells to begin expressing higher quantities of LTF peptides, glycosylating enzymes, or other proteins.

8. Identification and administration of drugs that can stimulate the activity levels of enzymes involved in creating higher quantities of fn/glyLTF.

Suppressing Deglycosylation and Degradation of fn/glyLTF

The second major category of potential treatments to increase inadequate levels of circulating fn/glyLTF involve methods and compounds that may be able to suppress the degradation of fn/glyLTF (such as by enzymes that strip away glycosyl moieties from fn/glyLTF). Candidate approaches that fall within this category include the following:

1. Extracorporeal treatment of blood, using reagents such as immobilized antibodies that bind to deglycosylating or other LTF-degrading enzymes. For example, blood from a patient who suffers from overly active deglycosylation of fn/glyLTF molecules can be extracted from a vein or artery (usually from an arm vein), and passed the blood through a column which contains monoclonal antibodies affixed to beads inside the column. The monoclonal antibodies, that are immobilized inside the column, will bind tightly to the over-abundant or over-active deglycosylating enzymes in the patient's blood. When that binding reaction occurs, the deglycosylating enzymes become trapped inside the column, and are thereby removed from the blood which emerges from the outlet of the column. The exiting blood is returned to the patient (usually into a vein in the patient's other arm, on a continuous processing basis, so that there is very little reduction in the patient's blood volume while the processing is being done).

Periodically, the flow of blood through the antibody column is interrupted, and a solution containing high levels of salt and/or acidity is passed through the column (often at a somewhat elevated temperature, as well). The elevated salt, acidity and/or temperature conditions inside the column will weaken the binding of the deglycosylating enzymes to the immobilized antibodies. This will allow the deglycosylating enzymes to be rinsed out of the column, thereby renewing the antibodies immobilized inside the column, so the column can be used again.

2. Identification, development, and administration of small-molecule drug compounds which can suppress or inactivate deglycosylating or other LTF-degrading enzymes. This type of suppression or inactivation is usually accomplished by using drugs that will bind tightly to the reaction site of an enzyme; when a drug binds to an enzyme reaction site for prolonged periods of time, that site is rendered unavailable for catalyzing biochemical reactions. Once the amino acid sequence and structure of a target enzyme is known, the development of enzyme-suppressing drugs can be greatly accelerated by computer modelling of candidate compounds.

3. Administration or other utilization of antibodies, peptide fragments, or other peptides or large molecules which can suppress the activity of deglycosylating or other LTF-degrading enzymes. One such approach involves the direct injection of antibodies, in a manner comparable to the types of antibody injections that are often used to prevent or treat flu or hepatitis infections. Such injections typically have a limited effective duration, such as roughly 30 days.

In an alternate approach which may be preferable for certain types of patients (such as, for example, morbidly obese patients who do not respond adequately to other treatments), it may be possible to inject into a patient an antigenic compound which contains a prominently exposed amino acid sequence that is identical to, or closely resembles, the reaction site of an overlay active or abundant enzyme which deglycosylates or otherwise degrades LTF. This type of antigenic treatment may provoke the patient's immune system to generate antibodies that will suppress, on a long-term basis, the degradation of fn/glyLTF. Clearly, an intervention which leads to a long-term alteration the patient's immune system will need to be carefully evaluated, since it poses a risk of long-term side effects. Nevertheless, this or similar approaches may offer useful treatments for some classes of patient.

4. Identification and administration of drugs or other compounds that can suppress the expression of genes which create enzymes that degrade fn/glyLTF. Such drugs might include compounds that have direct “gene suppressing” effects, and/or drugs that may be involved in one or more feedback circuits which instruct cells to suppress additional expression of enzymes that degrade fn/glyLTF.

5. Certain types of gene therapy may also be useful in suppressing the expression of enzymes that degrade fn/glyLTF. For example, an exogenous gene might be inserted which will express multiple copies of anti-sense mRNA strands, which can bind to mRNA strands which encode LTF-degrading enzymes. This type of double-stranded RNA cannot be expressed into proteins at normal rates or concentrations. Alternately, various techniques can be used to create “knockout” mutant cells containing a specific known gene that has been inactivated. Under certain conditions, such “knockout mutant” cells with one or more inactivated genes which no longer encode a deglycosylating or similar enzyme might be useful for reducing the levels of LTF-degrading enzymes circulating in the blood of a patient.

Development of “Surrogate” fn/glyLTF Molecules

The third category of potential treatments for patients who suffer from an inadequate or defective fn/glyLTF system involves the development of compounds which can function as “surrogate” forms of fn/glyLTF, by promoting the transport of leptin to and/or across the blood-brain barrier. Such compounds might include, for example:

1. Fragments or analogs of fn/glyLTF which do not require extensive glycosylation.

2. Fragments or analogs of fn/glyLTF which are not highly susceptible to degradation by enzymes that rapidly degrade complete fn/glyLTF molecules in blood

3. Drugs, peptides, or other compounds that can react with and activate membrane receptors that promote leptin transport across the BBB.

The analysis of and search for compounds that can serve as surrogates for fn/glyLTF is also likely to help stimulate and encourage the development of reagents, methods, and in vitro screening tests which can measure leptin transport across the BBB, or across membranes or tissues (including membranes created by tissue-culture methods) which are useful in in vitro tests. This type of work may also lead to highly useful screening tools and assays for use in such research.

Any of the treatment approaches outlines above, in conjunction with physician-supervised diet and exercise programs, can help promote improved approaches to weight loss and long-term weight control.

Additional Uses and Approaches

Several additional options and approaches should also be recognized, for potential use with one or more of the fn/glyLTF compounds or related methods outlines above.

One such approach involves direct injection, infusion, or similar administration of fn/glyLTF which is mixed with leptin, or with a surrogate form of leptin (such as a fragment or analog of the leptin polypeptide). In such a mixture, it is assumed that some portion of the leptin (or surrogate leptin) molecules would become reversibly bound to fn/glyLTF molecules, on an equilibrium basis, while in solution prior to injection. Such complexes may be able to increase and enhance the bioavailability and/or bioactivity of the leptin (or surrogate leptin) in such mixtures.

This invention further discloses methods of using fn/glyLTF in circulating blood as an indicator compound, for use in analyzing and diagnosing factors which contribute to impairments in fat metabolism and weight control, in obese patients. In particular, this invention includes the development of immunoassays, immunoblotting methods, and other assays and methods for analyzing fn/glyLTF concentrations in circulating blood, cerebrospinal fluid, or other body fluids or tissues. This invention also discloses the development of monoclonal or polyclonal antibody lines which bind to fn/glyLTF, and separate and distinct monoclonal or polyclonal antibody lines which bind to def/LTF, for use in such assays.

This invention also discloses preparations, reagents, testing kits, and methods which can specifically distinguish between fn/glyLTF and def/LTF, for use in testing “fa/fa” rats or other animals, or for testing blood or tissue from obese humans. Such kits and reagents offer highly useful tools and reagents for medical analyses that focus specifically on the roles of fn/glyLTF and def/LTF in fat metabolism, energy metabolism, and weight control. Typically, such kits will contain either of two sets of reagents. In one common embodiment, a kit will contain a combination of two different antibody preparations, wherein a first antibody preparation is capable of binding to glycosylated leptin transport factor. The other antibody preparation is capable of binding to leptin transport factor which is not fully glycosylated, but it does not bind to leptin transport factor which is fully glycosylated. In the other most common embodiment, a single antibody preparation is used, which is capable of binding to glycosylated leptin transport factor. The second reagent, instead of being an antibody preparation, is a leptin (or leptin ligand fragment) which will bind to glycosylated leptin transport factor, and which has been labelled to facilitate quantitative analysis in a diagnostic test. Various methods of labelling these types of ligands which will bind to antibodies are known to those skilled in the art, and include, for example, radiolabelled ligands, fluorescently-labelled ligands, chemoluminescent ligands, biotinylated ligands, and ligands coupled to enzymes (such as horseradish peroxidase) which will cause a colorimetric or other quantifiable reaction when mixed and incubated with suitable substrates or other reagents.

This invention also discloses that “fa/fa” rats (or other animals, including genetically manipulated animals with “knockout” mutations in either or both copies of their db genes) offer a highly useful animal model for further research on obesity, when tested using methods and reagents that can distinguish between fn/glyLTF and def/LTF.

### EXAMPLES

### Example 1

### Polyclonal Antibodies to LTF

An oligopeptide was synthesized by Research Genetics (Huntsville, Ala.) that corresponded with amino acids 473 through 488 of the LTF polypeptide (also called “Ob-Re” in the prior art). That amino acid sequence is CYSDIPSIHPISEPKD.

Ovalbumin was reacted with a 10-fold molar excess of sulfo-SMCC (sulfosuccinimidyl 4-[N-maleimidomethyl]-cyclohexane-1-carboxylate) (Pierce; Rockford, Ill.). Unbound sulfo-SMCC was removed with a G-50 desalting column. The resulting activated ovalbumin was conjugated to the synthetic polypeptide, via the sulfhydryl group on the N-terminal cysteine residue of the polypeptide.

Guinea pigs were injected at days 0, 30, and 60 with 0.5 mg of the ovalbumin-LTF conjugate, emulsified in Freund's adjuvant. Blood was sampled 14 days after the second boost (day 74), and subsequently 14 days after each boost. Blood was transferred to vacutainer tubes, allowed to clot for 30 min, and centrifuged to collect the serum containing anti-LTF antibodies.

### Example 2

### Western Blotting Procedures

Serum samples of 2-3 μl were mixed with identical volumes of a RIPA buffer (a 50 mM, pH 7.4; Tris-HCl solution containing 1% Nonidet P-40, 0.25% sodium deoxycholate, 150 mM NaCl, 1 mM EDTA, PMSF, Na₃VO₄ and NaF and 1 μg/ml each of aprotinin, leupeptin, and pepstatin) and a 2× volume of sample loading buffer (Bio-Rad Laboratories, Hercules Calif.) containing 2% B-mercaptoethanol. The mixtures were loaded onto 4-15% SDS-PAGE gels and processed using suitable voltages and times (typically 100 mV for about 90 minutes).

The separated proteins were electroblotted onto PVDF membranes (Millipore Co., Bedford, Mass.), using a transfer buffer containing 20% methanol. Membranes were then immunoblotted with anti-LTF serum (Example 1), or with an antibody that binds to leptin which had been diluted at 1:1000-5000 using blocking buffer (10 mM phosphate buffered saline (PBS) containing 5% non-fat milk and 0.1% Tween-20). Secondary antibodies which bind to IgG from guinea pigs (or rabbits), obtained from Amersham (Arlington Heights, Ill.), diluted at 1:12,000 (guinea pig) or 10,000 (rabbit), were labelled with horseradish peroxidase (Amersham). Protein bands were visualized by ECL Plus (Amersham) and autoradiography.

For non-denaturing gels, all processes were the same as described above, except that no SDS or β-mercaptoethanol were included during electrophoresis, and no methanol was contained in any transfer or rinsing buffer.

### Example 3

### Precipitation of LTF from Pooled Plasma

Two ml of Affi-Gel beads (Bio-Rad Labs) were poured into a Buchner funnel fitted with #4 Waterman filter paper. The beads were washed with cold water (distilled and deionized) until the alcohol smear disappeared. The beads were then transferred to a 15 ml conical tube. Two mg of rat or human leptin were added into the tube, and 10 mM PBS containing 0.1% sodium azide (as an antibacterial agent) was added until total volume was 5 ml.

After 4 hours of gentle agitation at 4° C., 1 ml of 1 M Tris-HCl was added. The tube was then gently agitated for another hour. The leptin-coupled beads were then washed 4 times in cold PBS. After the last wash with PBS, the beads were divided into two halves. Each aliquot was incubated with 5 ml pooled fresh plasma from either obese or lean Zucker rats, or from obese or lean humans. 7.5 ml of 10 mM PBS containing 0.1% sodium azide were used to dilute the plasma. Incubation continued overnight at 4° C., and beads were then washed three times in cold PBS.

### Example 4

### In Vitro Deglycosylation of LTF

The bead samples prepared as described in Example 3 were denatured by boiling in denaturing buffer, for 10 minutes. Each sample was divided into halves. One half was subdivided into aliquots which were deglycosylated, using an enzyme called PNGase F (New England Biolabs; Beverly, Mass.). Aliquots from the other half were used as controls.

15 μl supernatants that contained LTF released from leptin-bound beads were mixed with PNGase F reaction buffer and a protease inhibitor cocktail (Sigma Company; St. Louis, Mo.) to a final volume of 25 μl. The samples intended for deglycosylation were treated with 2 μl (500 units/μl) of PNGase F at 37° C. for 3, 6, or 9 hours.

At the end of the incubation, a double volume of sample buffer was added to the tube. After mixing, each sample (treated or control) was loaded onto a 4-15% gradient SDS-PAGE gel for analysis using Western blotting procedures as described in Example 2.

### Example 5

### fn/glyLTF and def/LTF from Lean and Obese FA/FA Rats

2 μl of blood serum were drawn from obese (fa/fa mutant) or lean Zucker rats. These samples were separated using 4-15% SDS-PAGE gels, and analyzed by Western blotting using the anti-LTF antibody described in Example 1. The results are shown in FIG. 1; lanes 1-4 hold samples from lean rats, while lanes 5-8 hold samples from obese fa/fa rats.

The uppermost band has a molecular weight of about 120 kilodaltons (kD), and is believed to be a larger and heavier glycosylated form of the LTF polypeptide. The middle band, with a molecular weight of about 70 kilodaltons (kD), is believed to be a smaller and lighter non-glycosylated (or less glycosylated) form of the same polypeptide. The lowest band, with a molecular weight of about 45 kD, is likely to be a degradation byproduct.

The bands in FIG. 1 make it clear that lean and healthy animals have substantially higher blood-borne concentrations of the glycosylated version (referred to herein as fn/glyLTF) of the leptin transport factor than obese fa/fa animals.

### Example 6

### Shift of LTF from Lean Rats After In Vitro Deglycosylation

Blood serum was tested from obese (fa/fa mutant) or lean Zucker rats. Half of the aliquots from each type of animal were deglycosylated in vitro, using PNGase F as described in Example 4. Treated and control samples were then separated on SDS-PAGE gels, followed by Western blotting using anti-LTF serum, using the procedures described in Example 5.

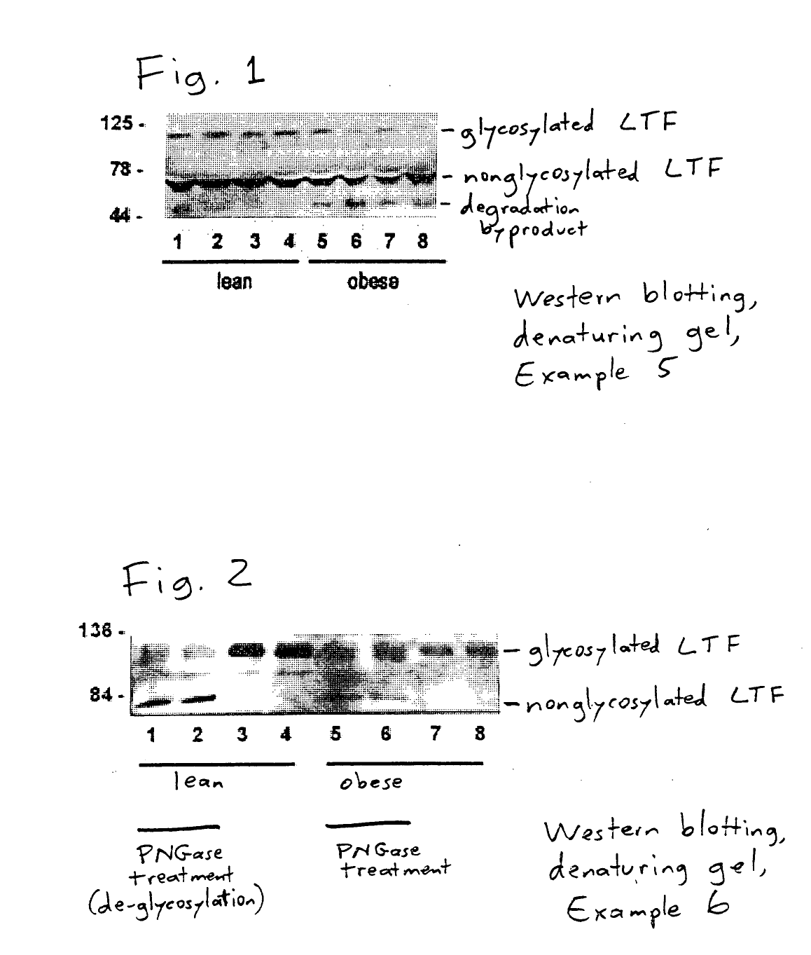

FIG. 2 shows the results, where Lanes 1-4 are samples from lean rats, while lanes 5-8 are samples from obese fa/fa rats. In these tests, the treated (deglycosylated) samples from the lean rats were loaded into lanes 1 and 2, while the untreated (control) samples from the lean rats were loaded into lanes 3 and 4. Similarly, lanes 5 and 6 show treated (deglycosylated) samples from obese rats, while lanes 7 and 8 show untreated (control) samples from obese rats.

The results in lanes 1 and 2 (lean rats, PNGase-treated samples) in FIG. 2 show a large shift of LTF protein down from the heavy upper band (fn/glyLTF, about 125 kD) into a smaller and lighter band of deglycosylated LTF (def/LTF, about 80 kD) due to enzymatic removal of glycosyl moieties. In lanes 3 and 4, the untreated control samples from lean rats did not move; these lanes show heavy bands of fn/glyLTF, and no significant levels of def/LTF.

In lanes 5-8 (samples from obese fa/fa rats), the bands of fn/glyLTF at about 125 kD are substantially fainter than in bands 1-4 (lean rats). In lanes 5 and 6, there is some degree of shift of LTF protein down from the heavy upper band (fn/glyLTF) into a smaller and lighter band of deglycosylated LTF (about 80 kD).

The fact that there is no clear detectable band of def/LTF (i.e., the presumably non-glycosylated or de-glycosylated version of LTF) in lanes 7 and 8 (obese rats) indicates that def/LTF is unstable in blood.

### Example 7

### Leptin Bound to LTF in Non-Denaturing Gels

Blood serum was drawn from both lean and obese rats. It was separated on non-denaturing gels, under conditions which allowed leptin molecules to remain bound to LTF molecules. In separate but parallel lanes, Western blotting was carried out using two different types of antibodies, which bound to either leptin (anti-LEP antibodies), or to the leptin transport factor (anti-LTF antibodies).

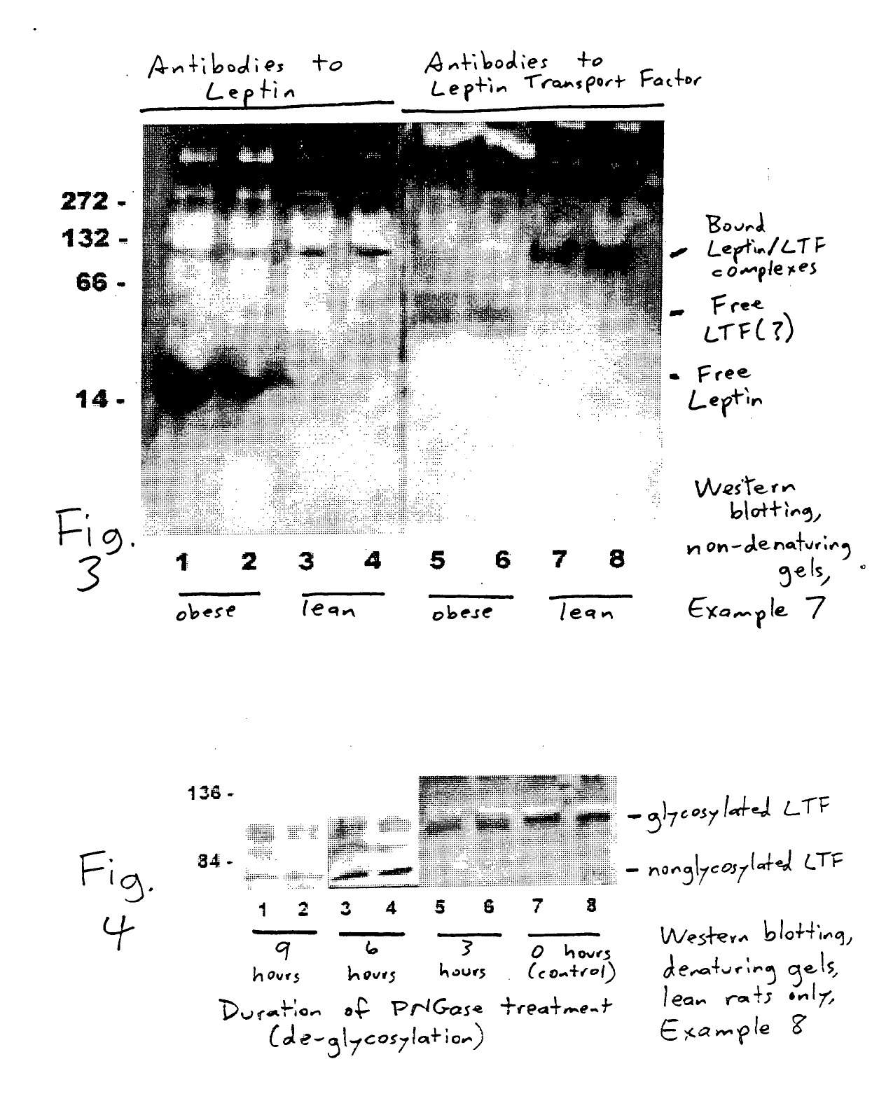

In FIG. 3, lanes 1 and 2 show serum from obese fa/fa rats, analyzed using anti-leptin antibodies. The very heavy band at about 16 kD shows free leptin molecules, which are not bound to LTF molecules. There is also a very faint band at about 120 kD, showing a small amount of leptin bound to LTF.

Lanes 3 and 4 show serum from lean rats, analyzed using the same anti-leptin antibodies. There is no substantial band showing any free and unbound leptin at 16 kD. However, there is a significant band showing leptin bound to LTF at about 120 kD.

Lanes 5 and 6 show serum from obese fa/fa rats, analyzed using anti-LTF antibodies. The fuzzy but significant bands at about 50 kD may be one or more degradation products that are created when non-glycosylated LTF begins to be digested and degraded by enzymes.

Lanes 7 and 8 show serum from lean rats, analyzed using anti-LTF antibodies. The bands at about 120 kD correspond to similar bands in lanes 3 and 4. This clearly indicates that the 120 kD complexes in lanes 3, 4, 7, and 8 consists of leptin molecules (bound to the anti-LEP antibodies in lanes 3 and 4) and LTF molecules (bound to the anti-LTF antibodies in lanes 7 and 8).

The obese rats show much higher levels of free leptin (lanes 1 and 2) than lean rats (lanes 3 and 4). However, the obese rats had very limited LTF-bound leptin as compared to lean animals. As in Example 6, the dominant form of LTF is the heavier glycosylated version found in lean animals, whereas most LTF in obese animals has a lower molecular weight, and is believed to have substantially lower levels of glycosylation.

### Example 8

### Time-Dependent Effects of PNGase Deglycosylation

A time-dependent study was carried out, to evaluate the effects of different treatment periods using the PNGase enzyme. This study used LTF extracted from pooled serum from lean rats by immunoprecipitation. Aliquots were treated by PNGase, using the methods described in Example 4, for 3, 6, or 9 hours. After each such treatment, the digested mixture was separated using SDS-PAGE, followed by Western blotting.

The results are shown in FIG. 4, where the control (untreated) samples are in lanes 7 and 8, which show heavy bands of fn/glyLTF at about 120 kD. Lanes 5 and 6 show somewhat lighter bands at 120 kD, following 3 hours of digestion using PNGase. Lanes 3 and 4 show substantially lighter bands at 120 kD, and the appearance of substantial bands of deglycosylated LTF at about 80 kD, following 6 hours of digestion using PNGase. Lanes 1 and 2 show only faint bands at both the 120 and 80 kD locations.

The presence of only faint bands at the 80 kD location in lanes 1 and 2, compared to the heavier bands in lanes 3 and 4, suggest that active degradation of the deglycosylated LTF polypeptide may have been occurring, before the digestion mixture was put onto the SDS-PAGE gel.

### Example 9

### Binding of Labelled Leptin to LTF

Additional tests were carried out using radiolabelled (¹²⁵I) leptin. Serum from lean or obese fa/fa rats was incubated with ¹²⁵I-leptin, then electrophoresed under non-denaturing conditions, so that any leptin which became bound to LTF would remain bound to it. The gels were then photographed.

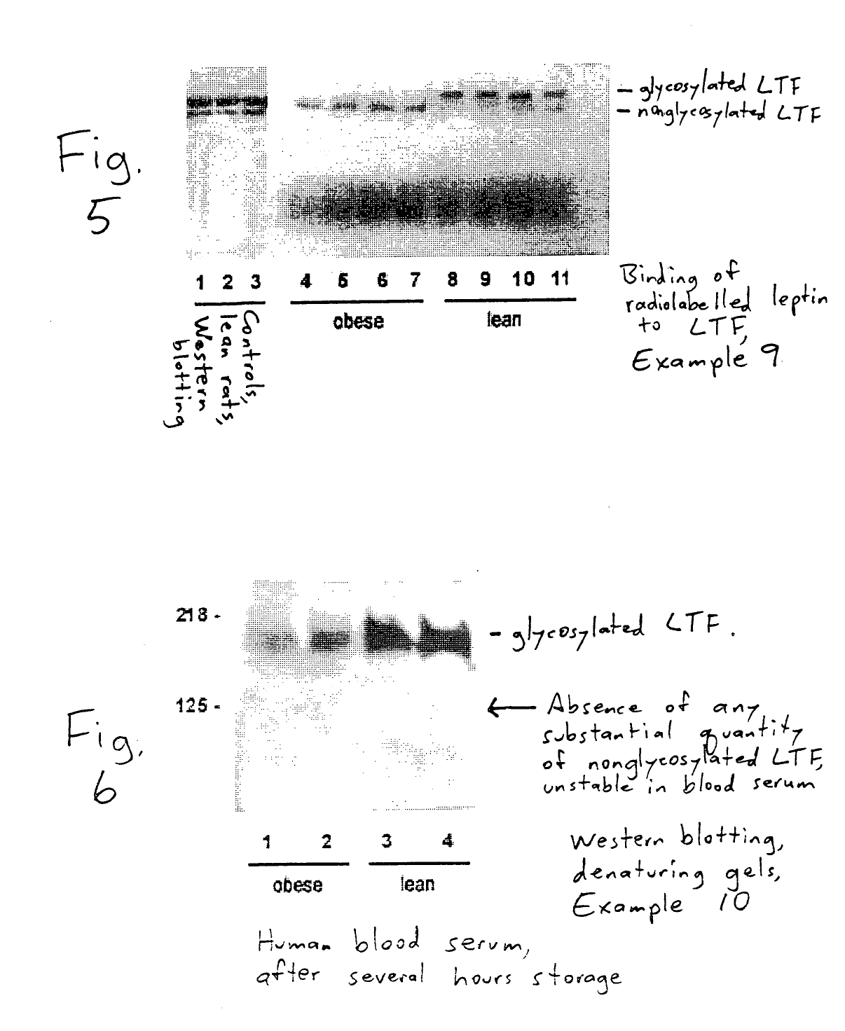

Control samples of serum from lean rats were not incubated with labelled leptin; instead, they were immunoblotted, using the Western blotting procedures described above. These controls, shown in lanes 1-3 of FIG. 5, indicate two distinct bands of LTF, with the upper band having heavier molecular weights (presumably due to higher levels of glycosylation) and the lower band having lower molecular weights (presumably due at least in part to lower or non-existent levels of glycosylation).

The serum from obese rats, in lanes 4-7 of FIG. 5, do not show two distinct bands of LTF. Instead, these lanes show only a single band, which migrates through the gels alongside the chemically-treated deglycosylated LTF bands from lean rats.

The serum from lean rats, in lanes 8-11, shows two distinct bands: a glycosylated form of LTF (the darker upper band), and a nonglycosylated form of LTF (the fainter lower band).

### Example 10

### Analysis of Human Serum, Lean and Obese

Samples of blood serum were obtained from lean and healthy human volunteers, and from obese patients undergoing medical care at an obesity clinic. Aliquots were processed on SDS-PAGE gels, and immunoblotted using the same anti-LTF antibodies described in Example 1.

In FIG. 6, lanes 1 and 2 show the samples from obese patients, while lanes 3 and 4 show the samples from lean and healthy volunteers.

Comparison of the bands at about 180 kD indicates that lean humans have much higher concentrations of the heavier fully-glycosylated form of fn/glyLTF than obese humans.

Also, it should be noted that these tests were not run on fresh blood samples; instead, they were run on samples that were several hours old. The absence of any lower bands with lower molecular weights, even in the blood samples from obese humans, indicates that the lighter (presumably deglycosylated or non-glycosylated) version referred to herein as def/LTF is relatively unstable and subject to digestion, and does not last long in blood serum.

Densitometric quantitation of fn/glyLTF levels were also carried out on blood samples from humans. A clear inverse correlation (probability less than 0.1%) was observed between body weight and fn/glyLTF concentrations in blood.

### Example 11

### Analysis of Month-Old Human Blood

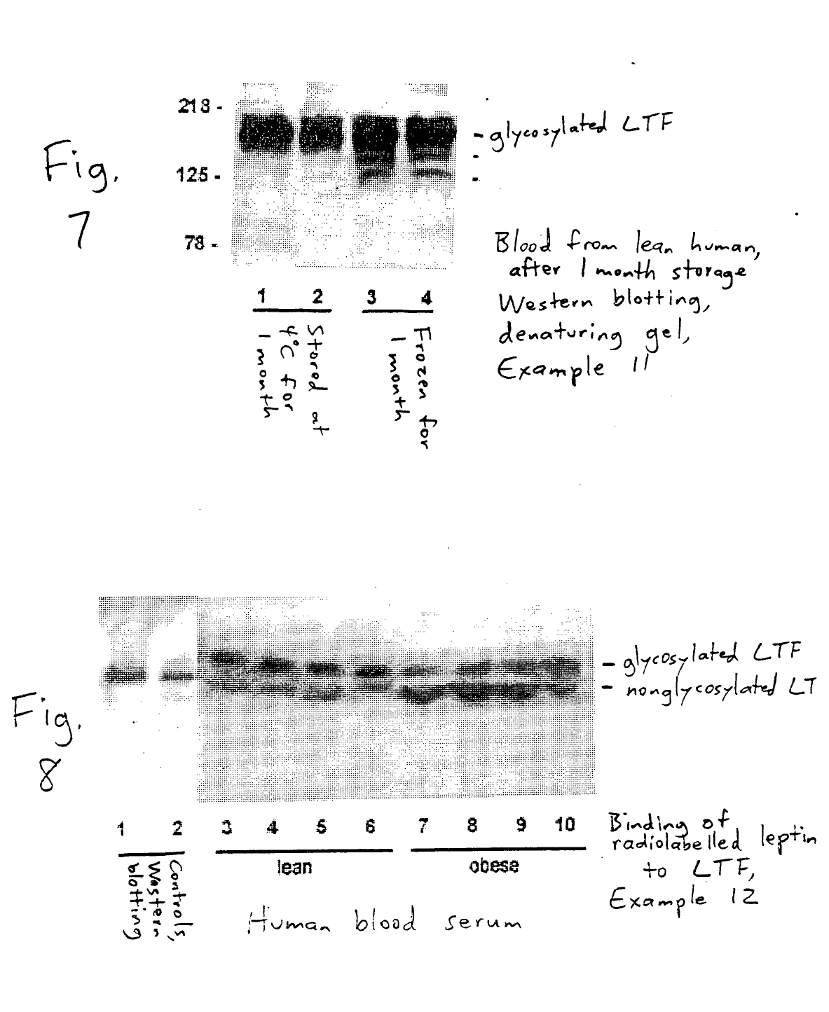

A sample of blood from a lean volunteer, with a normal concentration of fn/glyLTF, was divided into aliquots. Half were kept frozen for a month, and half were stored at 4° C. for a month. The aliquots were then separated on SDS-PAGE gels, followed by Western blotting. The results are shown in FIG. 7.

In this figure, lanes 1 and 2 show blood that was refrigerated at 4° C. for a month. There is a single large band, showing the glycosylated form of fn/glyLTF. However, there are no lower bands showing any detectable levels of def/LTF.

By contrast, lanes 3 and 4 show the results from blood that was kept frozen until shortly before processing. Two lower bands (at least one of which is believed to be a nonglycosylated version of LTF) are clearly visible, confirming that the blood sample did indeed contain def/LTF.

The disappearance of the def/LTF from the refrigerated (non-frozen) blood sample gives further confirmation that the def/LTF is relatively unstable, and is degraded by something (presumably proteolytic enzymes) that is naturally present in circulating blood.

### Example 12

### Radiolabelled Leptin Binding in Human Blood

Samples of blood were obtained from obese patients and lean volunteers. These samples were incubated with ¹²⁵I-labelled leptin and then processed on non-denaturing gels, using the methods described in Example 9. Control samples (lanes 1 and 2 in FIG. 8) were electrophoresed and then immunoblotted, as described above.

Blood samples from lean volunteers are shown in lanes 3-6, in FIG. 8. These lanes show a relatively heavy upper band of fn/glyLTF, and a fainter lower band on def/LTF.

Blood samples from obese patients are shown in lanes 7-10. These lanes show reversed results, with heavy lower bands indicating def/LTF, and fainter upper bands indicating fn/glyLTF.

### Example 13

### Creation and Immuno-Sequestered Implantation of Cells that Secrete fn/glyLTF

Mammalian cells that secrete fn/glyLTF can be created by genetic engineering of a host cell line which is known to secrete glycosylated proteins. One example a fibroblast cell line which has these traits, and which has been characterized in detail and is widely used in animal research, is the 3T3 cell line, described in articles such as Engelman et al 1999. Numerous other cell lines which also secrete glycosylated proteins are also known, including various human cell lines.

The selected cell line can be genetically transformed by a suitable vector, such as a plasmid (if multiple copies of the LTF-encoding gene are desired) or a virally-derived vector, such as a disarmed adenovirus vector. An example of an adenoviral vector which was modified to carry the Ob-Re coding sequence is described in Huang et al 2001.

The foreign gene(s) carried by the vector should be suitable for complete expression of a mature polypeptide, following any mRNA splicing or post-translational processing (to remove introns, leader sequences, etc.) in the selected host cells. The polypeptide encoded by the foreign gene preferably should be expressed at a relatively high level, under the control of a strong gene promoter, which might be either constitutive or inducible, depending on the needs of the patient. Depending on the nature and type of the LTF defect in a specific patient being treated, the polypeptide(s) encoded by the foreign gene can comprise the leptin transport factor polypeptide (or an effective fragment thereof), and/or a glycosidase enzyme which is known to add sugar moieties to the leptin transport factor polypeptide (such as N-glycosidase F).

After a population of cells has been treated with the vector, a clonal cell line which expresses and secretes a relatively high concentration of glycosylated LTF can be selected using a suitable screening technique, such as an assay that uses monoclonal antibodies which bind to LTF.

Cells from the selected cell line can then be embedded inside an “immuno-sequestered” (also called “immuno-isolated” or “immuno-privileged”) device which can be implanted surgically, using minimally invasive techniques such as a large-bore hypodermic needle which can be guided by a fluoroscope during the implantation procedure. Such devices typically are made of a non-resorbable porous biocompatible materal (often in the shape of a tube with sealed ends) which serves three functions: (i) it allows oxygen and nutrient molecules to reach the enclosed cells; (ii) it allows protein molecules secreted by the cells to emerge from the device and enter circulating blood or lymph; and, (ii) it prevents antibodies and immune cells from entering the device and commencing an immune response which would destroy the implanted cells. Such matrices and implants are described in patents such as U.S. Pat. Nos. 6,054,142 and 6,231,879 (both by Li et al, both entitled “Biocompatible devices with foam scaffolds”) and U.S. Pat. No. 5,773,286 (Dionne et al, entitled “Inner supported biocompatible cell capsules”), and in various other patents and articles cited therein.

This approach can be used to provide a surgically implanted source of fn/glyLTF from viable, living cells which have no specific limitation on how long they can continue to live and secrete fn/glyLTF inside a patient.

Thus, there has been shown and described a new discovery, showing that in at least some animals and humans suffering from obesity, an unstable low-molecular-weight version of the leptin transport factor poses a potentially crucial yet treatable defect in dysfunctional leptin regulatory systems. Although this invention has been exemplified for purposes of illustration and description by reference to certain specific embodiments, it will be apparent to those skilled in the art that various modifications, alterations, and equivalents of the illustrated examples and embodiments are possible.

### REFERENCES

Alberts, B., et al, Molecular Biology of the Cell (Garland Publishing, New York)
Clement, K., et al, “A mutation in the human leptin receptor gene causes obesity and pituitary dysfunction,” Nature 392: 398-401 (1998)
Coleman, D. L., “Obese and diabetes: two mutant genes causing diabetes-obesity syndromes in mice,” Diabetologia 14: 141-148 (1978)
Collins, S., et al, “Role of leptin in fat regulation,” Nature 380: 677 (1996)
Considine, R. V., et al, “Leptin: Genes, concepts, and clinical perspective,” Hormone Research 46: 249-256 (1996)
Considine, R. V., et al, “Serum immunoreactive leptin concentrations in normal-weight and obese humans,” N. Engl. J. Med. 334: 292-295 (1996)
Engelman, J. A., et al, “Constitutively active mitogen-activated protein kinase kinase 6 (MKK6) or salicylate induces spontaneous 3T3-L1 adipogenesis,” J. Biol. Chem. 274: 35630-38 (1999)
Friedman, J. M., et al, “Leptin and the regulation of body weight in mammals,” Nature 395: 763-70 (1998)
Halaas, J., et al, “Weight reducing effects of the plasma protein encoded by the ob gene,” Science 269: 543-546 (1995)
Hanui, M., et al, “Human leptin receptor: Determination of disulfide structure and N-glycosylation sites of the extracellular domain,” J. Biol. Chem. 273: 28691-28699 (1998)
Huang, L., et al, “Modulation of circulating leptin levels by its soluble receptor,” J. Biol. Chem. 276: 6343-6349 (1998)
Montague, C. T., et al, “Congenital leptin deficiency is associated with severe early-onset obesity in humans,” Nature 387: 903-908 (1997)
Pelleymounter, M. A., et al, “Effects of the obese gene product on body weight regulation in ob/ob mice,” Science 269: 540-543 (1995)
Schwartz, M., et al, “Specificity of leptin action on elevated blood glucose levels and hypothalamic neuropeptide Y gene expression in ob/ob mice,” Diabetes 45: 531-535 (1996)
Spiegelman, B. M., et al, “Adipogenesis and obesity: rounding out the big picture,” Cell 87: 377-89 (1996)
Tartaglia, L. A., et al, “Identification and expression cloning of the leptin receptor, OB-R,” Cell 83: 1263-1271 (1995)
Zhang, y., et al, “positional cloning of the mouse obese gene and its human homologue,” Nature 372: 425-432 (1994)

1. A method for improved pharmacologic control of fat metabolism and body mass in mammals, comprising the step of introducing, into circulating blood of a mammal being treated, a pharmacologic preparation containing glycosylated leptin transport factor (also known as Ob-Re or sOb-R), in a therapeutic quantity which induces weight loss in the mammal being treated.

2. The method of claim 1 wherein the glycosylated leptin transport factor is introduced into the circulating blood by means selected from the group consisting of intravenous injection, intravenous infusion, and intramuscular injection.

3. The method of claim 1 wherein the glycosylated leptin transport factor is introduced into the circulating blood by subcultaneous implantation of a sustained-release device containing glycosylated leptin transport factor.

4. The method of claim 1 wherein the glycosylated leptin transport factor is introduced into the circulating blood by implantation of a device which contains cells that secrete glycosylated leptin transport factor, enclosed within a permeable encapsulating material that prevents an immune rejection response in the body of the mammal being treated.

5. The method of claim 4, wherein the cells that secrete glycosylated leptin transport factor have been genetically engineered to contain at least one exogenous gene which encodes a polypeptide selected from the group consisting of a leptin transport factor, a portion of a leptin transport factor which after glycosylation binds to leptin in circulating blood, and a glycosidase enzyme which adds sugar moieties to a leptin transport factor of polypeptide.

6. The method of claim 1 wherein the glycosylated leptin transport factor is introduced into the circulating blood by means selected from the group consisting of trans-membrane permeation, and oral ingestion of a capsule having an enteric coating that resists digestion by stomach acid.

7. A method of genetic treatment to induce weight loss in a mammal, comprising the following steps:
a. removing, from the mammal, at least one selected cell type which can generate large numbers of progency cells that will be capable of secreting glycosylated proteins;
b. treating the selected cell type with a genetic vector which carries at least one foreign gene which encodes a polypeptide selected from the group consisting of a leptin transport factor, and a portion of a leptin transport factor which, after glycosylation, will bind to leptin in circulating blood;
c. selecting progeny cells which have been genetically transformed, which express the leptin transport factor or portion thereof which is encoded by the foreign gene, and which secrete glycosylated copies of the leptin transport factor or portion thereof;
d. implanting the genetically transformed cells into the mammal being treated.

8. The method of claim 7, wherein the genetic vector also carries at least one foreign gene which encodes a glycosidase enzyme which is known to add sugar moieties to the leptin transport factor polypeptide.

9. A method of extracorporeal blood treatment to induce weight loss in mammals, comprising the following steps:
a. analyzing blood from a mammal suffering from excess weight, to identify at least one blood-borne enzyme which degrades glycosylated leptin transport factor;
b. selecting an antibody preparation which binds to a targeted blood-borne enzyme which degrades glycosylated leptin transport factor;
c. removing blood from the mammal;
d. passing the blood through a device which contains the selected antibody preparation, under conditions which enable the antibody preparation to contact the blood and remove a quantity of the targeted blood-borne enzyme which degrades glycosylated leptin transport factor; and,
e. returning the blood which has been passed through the device to the mammal being treated.

10-12. (cancelled)
